(19) United States
(12) Patent Application Publication
(10) Pub. No.: US 2019/0274864 A1
(43) Pub. Date: Sep. 12, 2019

(54) CERVICAL SPINE BRACE

(71) Applicant: Woo-yeol SHIN, Seoul (KR)

(72) Inventor: Woo-yeol SHIN, Seoul (KR)

(21) Appl. No.: 16/336,809

(22) PCT Filed: Sep. 27, 2017
(86) PCT No.: PCT/KR2017/010718

§ 371 Date: Mar. 26, 2019

(30) Foreign Application Priority Data
Oct. 13, 2016 (KR) 10-2016-0132942

(51) Int. Cl. A61F 5/04; A61H 1/02; A61H 39/04

(52) U.S. Cl. CPC A61F 5/04; A61H 1/0296; A61H 2201/1695; A61H 2201/1609; A61H 2205/04; A61H 39/04

(57) ABSTRACT

Provided is a cervical spine brace which transfers optimal stimulation to each portion of the cervical spine so as to enable more efficient correction. The cervical spine brace comprises: a support block disposed to be placed on the back of the neck between the head and the back and having an arch part corresponding to the arch of the back of the neck; and a plurality of pressing plates which are connected in pairs at opposite sides of the arch part of the support block, make an inclination toward the back of the neck from the support block, and have one or more edges at end portions thereof, the edges being arranged to form pressure points on different spots of the back of the neck depending on height of the arch part.

### TECHNICAL FIELD

The present invention relates to a cervical spine brace, and more particularly, to a cervical spine brace capable of transferring optimum stimulus to each region of the cervical vertebrae to realize more effective correction.

### BACKGROUND ART

Respective regions of the human body may cause unbalance for various reasons. Such unbalance may be caused by excessive exertion or injury, for example, or the unbalance of the body may occur when a person repeats a certain action or due to poor posture. In particular, for example, as in the case where the muscles of the head, the neck, and the back are strained and the vertebrae are deformed due to the life pattern of looking at a TV, a monitor, a mobile device, etc. for a long time, people are increasingly complaining of chronic fatigue or pain due to the unbalance of the body.

In order to solve this, various types of correction devices have conventionally been manufactured. A correction device that is developed for medical use is capable of applying strong pressure to the cervical vertebrae, the thoracic vertebrae, the lumbar vertebrae, etc. in a mechanical manner, but requires a skillful manipulation technique, and therefore ordinary persons have difficulty using the correction device and serious side effects may be caused when the correction device is incorrectly used. For this reason, for example, as disclosed in Korean Patent Laid-Open Publication No. 10-2016-0105247, a correction device has been manufactured in the form of a corrective pillow or a wooden pillow, for example, which may be easily used at home, and such a device is increasingly used to obtain correction effects.

However, most conventional correction devices have been configured to simply change the posture of the neck and the periphery thereof using a groove, for example. That is, in most cases, the correction devices serve to forcibly maintain the posture of the head or the neck, for example, and change the position of the deformed cervical vertebrae using a groove structure in which the head or the neck is accommodated. However, such a structure has a limitation in effectively stimulating the cervical vertebrae, thus resulting in insignificant correction effects. For example, the correction devices may transfer stimulus only to a specific region of the cervical vertebrae without stimulation on the other region according to the shape of a groove, or may adversely cause pain due to unnecessary posture correction. In addition, for example, a protrusion structure for acupressure treatment has been added, but it was difficult to fundamentally improve correction effects.

### PRIOR ART DOCUMENT

### Patent Document

Specification of Korean Patent Laid-Open Publication No. 10-2016-0105247 (2016.09.06)

### DISCLOSURE

### Technical Problem

A technical object of the present invention is to provide a cervical spine brace capable of transferring optimum stimulus to each region of the cervical vertebrae to realize more effective correction.

The technical objects of the present invention are not limited to the objects as mentioned above, and other unmentioned technical objects will be clearly understood by those skilled in the art from the following description.

### Technical Solution

In accordance with an aspect of the present invention, provided is a cervical spine brace including a support block configured to be disposed on a back of a neck between a head and a back and having an arch portion corresponding to an arch of the back of the neck, and a plurality of pressure plates connected in a pair to opposite sides of the arch portion of the support block and configured to be inclined from the support block toward the back of the neck, each pressure plate having at least one edge disposed on an end thereof to form pressure points at different positions on the back of the neck according to a height of the arch portion.

The arch portion may include a curved surface that is convex toward the back of the neck.

At least a portion of each pressure plate may be formed higher than the arch portion so that the back of the neck is inserted between the pressure plate and the arch portion.

The edge may be located on the end of the pressure plate located higher than the arch portion.

At least a portion of each pressure plate may have an arch-shaped rim, and the edge may be formed into a curved surface on the end of the pressure plate.

The at least one edge may include a first edge that extends upward from the arch portion and is interposed between the back of the neck and the arch portion and a second edge that extends outward of the first edge and is located higher than the first edge.

The first edge may be disposed adjacent to a spinous process of a cervical vertebra to be able to transfer pressure to the spinous process, and the second edge may be disposed adjacent to a transverse process of the cervical vertebra to be able to transfer pressure to the transverse process.

The first edge and the second edge may be formed respectively into curved surfaces having different centers of curvature.

The first edge may be formed into a curved surface that is shorter than the second edge.

The cervical spine brace may further include a first cervical vertebra pressure member having at least a portion disposed at a position corresponding to a first cervical vertebra, the first cervical vertebra pressure member protruding from the support block, the pressure plates may include a second cervical vertebra pressure plate disposed at a position corresponding to a second cervical vertebra, a third cervical vertebra pressure plate disposed at a position corresponding to a third cervical vertebra, a fourth cervical vertebra pressure plate disposed at a position corresponding to a fourth cervical vertebra, and a fifth cervical vertebra pressure plate disposed at a position corresponding to a fifth cervical vertebra, and the first cervical vertebra pressure member may have a size smaller than a size of the pressure plates.

The cervical spine brace may be configured such that the fourth cervical vertebra pressure plate has an uppermost end located higher than an uppermost end of the fifth cervical vertebra pressure plate, the second cervical vertebra pressure plate has an uppermost end located lower than an uppermost end of the third cervical vertebra pressure plate, and the first cervical vertebra pressure member has an uppermost end located lower than the uppermost end of the second cervical vertebra pressure plate.

In the cervical spine brace, the first cervical vertebra pressure member may have a pressure portion formed into a curved surface that extends upward from the arch portion, and each of the second cervical vertebra pressure plate, the third cervical vertebra pressure plate, the fourth cervical vertebra pressure plate, and the fifth cervical vertebra pressure plate may have two or more edges including one edge formed into a curved surface that extends upward from the arch portion and another edge formed into another curved surface.

The curved surface forming the pressure portion may have a length greater than a length of the one edge formed into the curved surface that extends upward from the arch portion.

In the cervical spine brace, at least one of a distance between the first cervical vertebra pressure member and the second cervical vertebra pressure plate, a distance between the second cervical vertebra pressure plate and the third cervical vertebra pressure plate, a distance between the third cervical vertebra pressure plate and the fourth cervical vertebra pressure plate, and a distance between the fourth cervical vertebra pressure plate and the fifth cervical vertebra pressure plate may be greater or smaller than another distance.

At least a portion of the first cervical vertebra pressure member may extend between a pair of the second cervical vertebra pressure plates.

Each pressure plate may be inclined in a direction corresponding to a direction in which a spinous process of a cervical vertebra extends.

Each pressure plate may be connected in a cantilever form to the support block.

The cervical spine brace may further include a pressure protrusion that protrudes from a top of the arch portion and is able to form a pressure point wider than the edge on the back of the neck that does not come into contact with the edge.

The pressure protrusion may have at least a portion disposed at a position corresponding to a sixth cervical vertebra and a seventh cervical vertebra, and may include an inclined contact surface to transfer pressure to both the sixth cervical vertebra and the seventh cervical vertebra via the contact surface.

The pressure protrusion may have at least a portion that extends between a pair of the pressure plates.

The cervical spine brace may further include a headrest connected to the support block.

The cervical spine brace may further include a support pad detachably coupled to a head contact portion of the headrest.

The cervical spine brace may further include a connection block configured to interconnect and secure lower ends of the pressure plates.

### Advantageous Effects

As apparent from the foregoing, the present invention advantageously provides a cervical spine brace capable of transferring optimum stimulus to each region of the cervical vertebrae to realize more effective correction. In particular, the cervical spine brace is configured to be able to transfer pressure to individual regions of the cervical vertebrae and to three-dimensionally apply stimulus to the cervical vertebrae in different directions, such as the transverse direction and the longitudinal direction, thus resulting in maximum correction effects. In addition, the cervical spine brace is capable of forming a pressure point corresponding to the cervical vertebrae to apply more effective stimulus and is also capable of transferring stimulus to various regions of the neck, thereby effectively eliminating and alleviating pain generated in a part or the entirety of the neck.

### DESCRIPTION OF DRAWINGS

FIG. 1 is a perspective view of a cervical spine brace according to an embodiment of the present invention;

FIG. 2 is a front view of the cervical spine brace of FIG. 1;

FIG. 3 is a plan view of the cervical spine brace of FIG. 1;

FIG. 4 is a side view of the cervical spine brace of FIG. 1;

FIG. 5 is a cross-sectional view of the cervical spine brace of FIG. 1;

FIG. 6 is a view illustrating one of pressure plates of the cervical spine brace of FIG. 1;

FIGS. 7 and 8 are views illustrating the use state of the cervical spine brace of FIG. 1;

FIG. 9 is a perspective view of a cervical spine brace according to another embodiment of the present invention;

FIG. 10 is a side view of the cervical spine brace of FIG. 9;

FIG. 11 is a view illustrating the use state of the cervical spine brace of FIG. 9;

FIG. 12 is a perspective view of a cervical spine brace according to a further embodiment of the present invention;

FIG. 13 is a front view of the cervical spine brace of FIG. 12; and

FIG. 14 is a side view of the cervical spine brace of FIG. 12.

[symbol]

### MODE FOR INVENTION

The advantages and features of the present invention and the ways of attaining the same will become apparent with reference to embodiments described below in detail in conjunction with the accompanying drawings. The present invention, however, is not limited to the embodiments disclosed hereinafter and may be embodied in many different forms. Rather, these exemplary embodiments are provided so that this disclosure will be through and complete and will fully convey the scope to those skilled in the art. The scope of the present invention should be defined by the claims. The same elements are denoted by the same reference numerals throughout the specification.

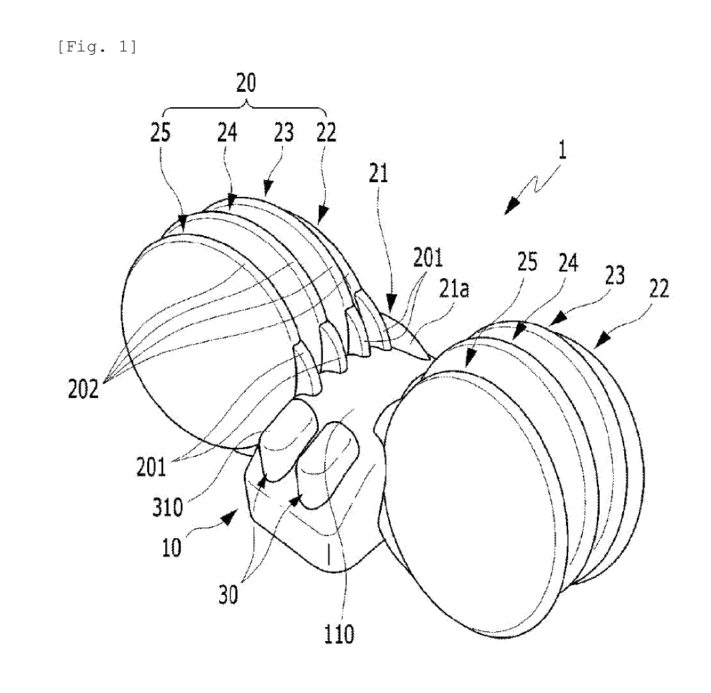

Hereinafter, a cervical spine brace according to an embodiment of the present invention will be described in detail with reference to FIGS. 1 to 8.

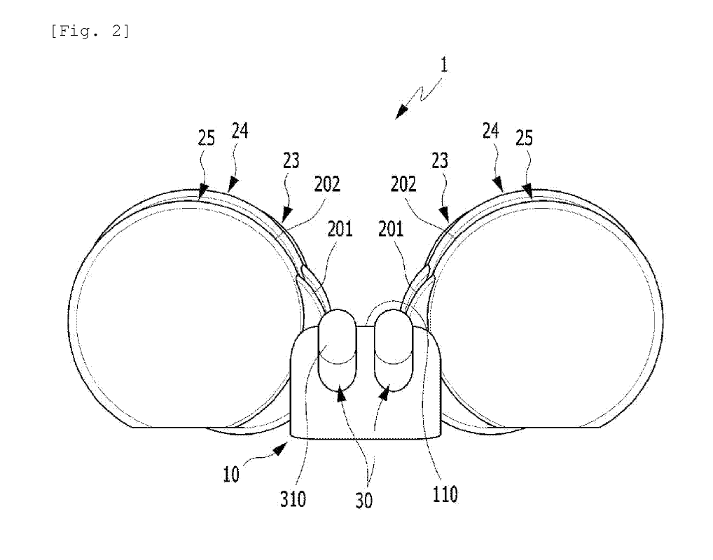

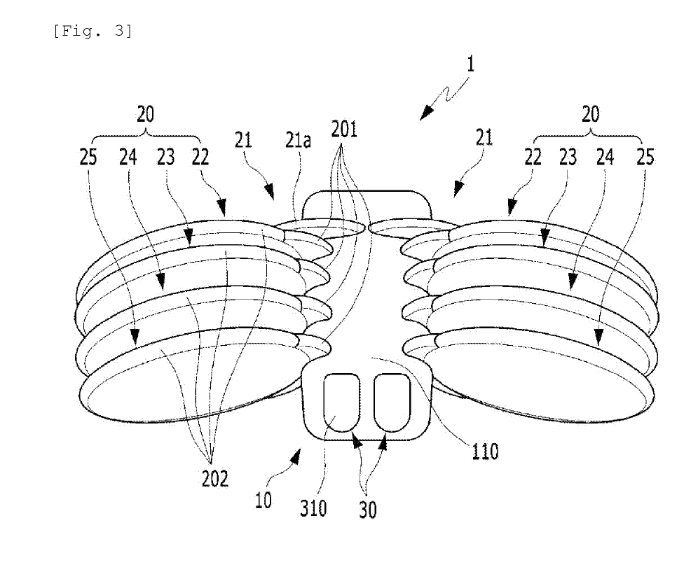

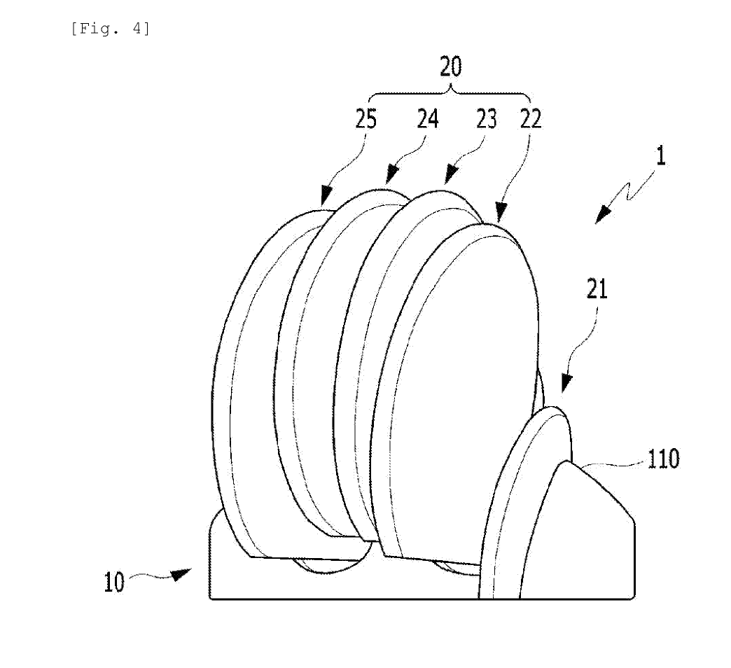

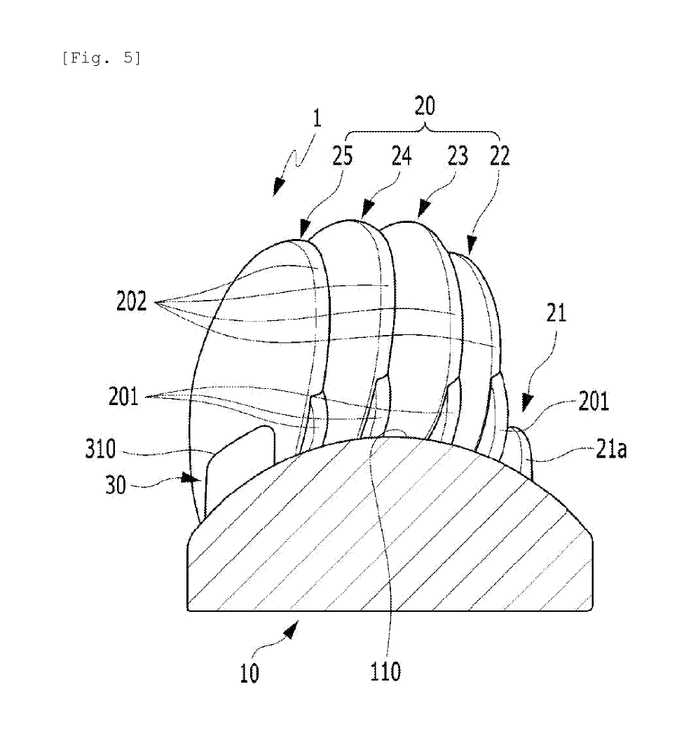

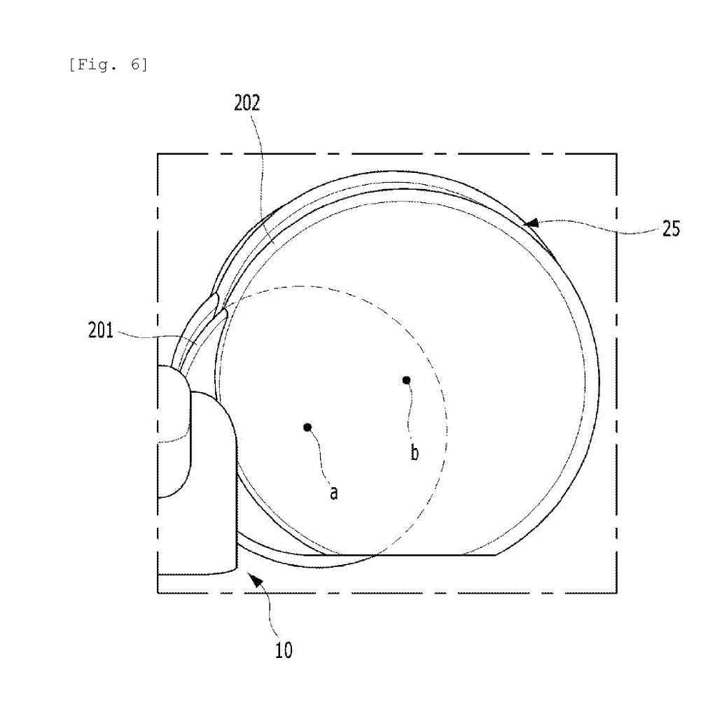

FIG. 1 is a perspective view of a cervical spine brace according to an embodiment of the present invention, FIG. 2 is a front view of the cervical spine brace of FIG. 1, FIG. 3 is a plan view of the cervical spine brace of FIG. 1, FIG. 4 is a side view of the cervical spine brace of FIG. 1, FIG. 5 is a cross-sectional view of the cervical spine brace of FIG. 1, and FIG. 6 is a view illustrating one of pressure plates of the cervical spine brace of FIG. 1.

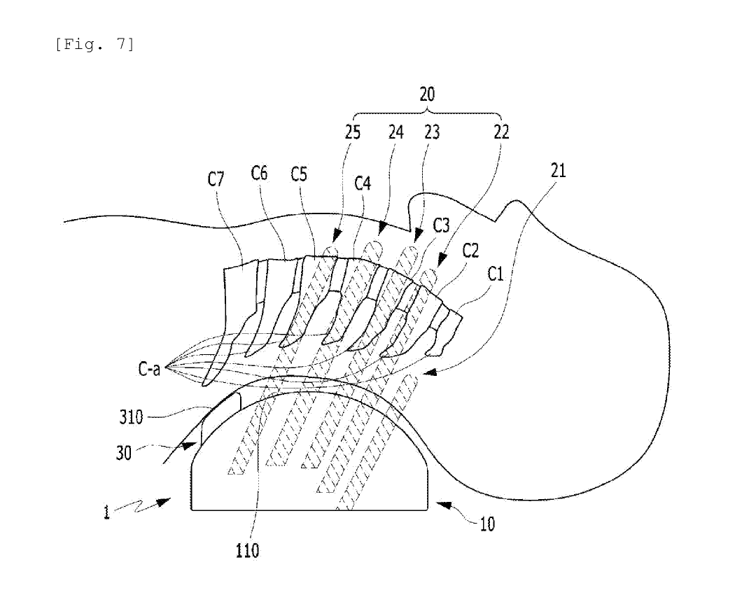

Referring to FIGS. 1 to 4, the cervical spine brace 1 according to an embodiment of the present invention includes a support block 10, which is configured to be disposed on the back of the neck between the head and the back (see FIG. 7) and has an arch portion 110 corresponding to the arch of the back of the neck, and a plurality of pressure plates 20, which are connected in pairs to opposite sides of the arch portion 110 of the support block 10 and are configured to be inclined from the support block 10 toward the back of the neck, each pressure plate having one or more edges 201 and 202 disposed on an end thereof so as to form pressure points at different positions on the back of the neck according to the height of the arch portion 110. The cervical spine brace 1 is configured such that the pressure plates 20, formed with the edges 201 and 202, extend in opposite directions of the support block 10. Here, the arch portion 110 may be located at the side opposite to the bottom surface of the support block 10 which is in contact with the floor, and the height of the arch portion 110 may be measured from the bottom surface of the support block 10. That is, for example, in the state illustrated in FIGS. 4, 5 and 7, the bottom surface of the support block 10 may be a lower surface, the arch portion 110 may be formed on an upper surface opposite to the lower surface, and the height of the arch portion 110 may be a straight distance from the lower surface to the upper surface.

The cervical spine brace 1 accommodates a pressure target region between the plurality of pressure plates 20 to very effectively apply pressure to the pressure target region. The pressure target region may be the neck where the cervical vertebrae (or the cervical spine) are disposed, and in particular, may be the back of the neck where no uvula is located. The arch portion 110 may form an arch corresponding to the arch of the back of the neck (see FIG. 7), and may include a curved surface which is convex toward the back of the neck as illustrated. Here, the curved surface of the arch portion 110 may be any of various convexly-curved surfaces having a symmetrical or asymmetrical shape. The pressure plates 20 are configured to effectively transfer pressure to the back of the neck using the edges 201 and 202 formed on the end thereof. Through the provision of the pressure plates 20 three-dimensionally arranged along the arch portion 110 and the edges 201 and 202 of the pressure plates 20, it is possible to apply pressure to a contact region in different directions, such as the longitudinal direction and the transverse direction. In addition, since the plurality of pressure plates 20 stimulate different cervical vertebrae at different positions, respectively, and transfer pressure to the periphery thereof, it is possible to easily correct deformation of the cervical vertebrae and to easily eliminate the tension and pain of muscles, for example.

Hereinafter, the cervical spine brace 1 according to the embodiment of the present invention having the features described above will be described in more detail with reference to the respective drawings. Other features not mentioned in the following description will be clearly understood.

The support block 10 includes the arch portion 110. The support block 10 is connected to the pressure plates 20, as illustrated in FIGS. 1 to 4. The support block 10 serves as a support body that secures and supports the pressure plates 20. In addition, the support block 10 may also serve to determine the arrangement of the pressure plates 20 via the arch portion 110. The arch portion 110 protrudes toward the back of the neck between the head and the back (see FIG. 7), and is formed into an arch shape corresponding to the arch of the back of the neck. The arch portion 110 may protrude in the form of a convexly curved surface. As illustrated in FIGS. 1 to 4, the arch portion 110 may protrude in an arch shape toward the back of the neck. The support block 10 takes the form of a block having the arch portion 110 described above.

The arch portion 110 of the support block 10 may be formed at the side opposite to the bottom surface by which the support block 10 is supported (i.e., the surface formed on a lower end portion of the support block in FIGS. 2 and 4). The arch portion 110 may be integrally formed with the support block 10. As illustrated in FIGS. 2 and 3, at least a portion of the arch portion 110 may be located between the pressure plates 20 to interconnect and secure the pressure plates 20. In addition, at least a portion of the arch portion 110 may be in direct contact with the back of the neck of a user and serve to support the back of the neck. The arch portion 110 may form a strong support structure by protruding in an arch shape toward the back of the neck. The protruding shape of the arch portion 110 may be appropriately adjusted as needed, and the protruding shape of the arch portion 110 will not be limited to the illustrated shape.

The pressure plates 20 are connected in pairs to opposite sides of the arch portion 110 of the support block 10. The plurality of pressure plates 20 are connected to different positions of the support block 10 to independently transfer stimulus to different cervical vertebrae. In addition, a protruding structure (a first cervical vertebra pressure member 21) which protrudes from the support block 10 may be used, along with the pressure plates 20, to effectively stimulate the cervical vertebrae. According to the embodiment of the present invention, the cervical spine brace 1 includes the first cervical vertebra pressure member 21 which protrudes from the support block 10 and is disposed at a position at which at least a portion thereof corresponds to the first cervical vertebra (see “C1” in FIG. 7), and the pressure plates 20 include a second cervical vertebra pressure plate 22 disposed at a position corresponding to the second cervical vertebra (see “C2” in FIG. 7), a third cervical vertebra pressure plate 23 disposed at a position corresponding to the third cervical vertebra (see “C3” in FIG. 7), a fourth cervical vertebra pressure plate 24 disposed at a position corresponding to the fourth cervical vertebra (see “C4” in FIG. 7), and a fifth cervical vertebra pressure plate 25 disposed at a position corresponding to the fifth cervical vertebra (see “C5” in FIG. 7). The size of the first cervical vertebra pressure member 21 may be smaller than the size of the other pressure plates 20.

That is, by using the plurality of pressure plates 20, it is possible to easily transfer pressure to different cervical vertebrae and the periphery thereof from the ends of the respective pressure plates 20. In addition, it is possible to easily transfer pressure to different cervical vertebrae and the periphery thereof by using the pressure plates 20 and the first cervical vertebra pressure member 21 together. In particular, by applying stimulus with the ends of the pressure plates 20 having a flat plate shape, it is possible to concentrate pressure on a narrow portion of the end to realize more effective transfer of the pressure. In addition, it is possible to more effectively transfer pressure via a pressure portion 21a formed on the first cervical vertebra pressure member 21. The pressure plates 20 are obliquely arranged from the support block 10 toward the back of the neck (see FIGS. 1 to 4 and 7), and respectively include one or more edges 201 and 202 disposed on the end thereof so as to form pressure points at different positions on the back of the neck according to the height of the arch portion 110 of the support block 10. In particular, the pressure plates 20 form three-dimensional pressure points, which come into contact with different positions on the back of the neck to transfer pressure thereto, via the edges 201 and 202 on the end thereof. A process of transferring pressure via the edges 201 and 202 and the pressure portion 21a, for example, will be described below in more detail.

Each pressure plate 20 may be formed with one or more edges 201 and 202. In addition, the first cervical vertebra pressure member 21 may be formed with the pressure portion 21a, which is formed into a curved surface extending upward from the arch portion 110. According to the embodiment of the present invention, each of the second cervical vertebra pressure plate 22, the third cervical vertebra pressure plate 23, the fourth cervical vertebra pressure plate 24, and the fifth cervical vertebra pressure plate 25 is formed with two or more edges 201 and 202, which include one edge (i.e., a first edge 201) formed into a curved surface extending upward from the arch portion 110 and another edge (i.e., a second edge 202) formed of another curved surface. Through the use of the edges 201 and 202 formed on the ends of the different pressure plates 20 as described above, it is possible to three-dimensionally apply stimulus to respective regions of different cervical vertebrae and the periphery thereof.

At least a portion of each pressure plate 20 is located higher than the arch portion 110 (see FIGS. 2 and 4). Thus, a certain accommodating space may be defined between the pressure plates 20 and the arch portion 110 so that the back of the neck may be easily inserted into the space between the pressure plates 20 and the arch portion 110. The accommodating space is formed into a three-dimensional shape corresponding to the shape of the arch portion 110 and the shape of the pressure plates 20 connected to opposite sides of the arch portion 110, for example. That is, as illustrated in FIGS. 1 to 3, by forming the accommodating space defined as a three-dimensional space between the convex arch portion 110, the plurality of pressure plates 20 arranged at opposite sides of the arch portion 110 along the arch portion, and the edges 201 and 202 formed on the pressure plates 20, it is possible to insert the pressure target region (i.e., the back of the neck) into the accommodating space and easily stimulate the pressure target region.

Since the arch portion 110 and the pressure plates 20 are arranged around the back of the neck, a three-dimensional pressure point (i.e., a point to which pressure is transferred, but not necessarily having a point shape and may have a planar shape) is formed, in particular, by the edges 201 and 202 formed on the pressure plates 20. In addition, since the gap between the respective pressure plates 20 is open, a so-called relaxation point (i.e., a point at which relaxation occurs, but not necessarily having a point shape and may have a planar shape) is formed between the pressure points to naturally relax the muscles and a connection region of the cervical vertebrae, for example. Accordingly, the cervical spine brace 1 of the present invention may induce appropriate stimulation and relaxation together. In this way, it is possible to maximize, for example, correction effects of the respective cervical vertebrae and the periphery thereof and the removal of pain therein. This will be described below in more detail.

As illustrated in FIGS. 1 to 3, the edges 201 and 202 are located on the end of each pressure plate 20 located higher than the arch portion 110. That is, as described above, at least a portion of the pressure plate 20 may be located higher than the arch portion 110 in the direction in which the arch portion 110 protrudes, and the edges 201 and 202 may be disposed on the end of the pressure plate located higher than the arch portion 110 to easily form pressure points. At least a portion of the pressure plate 20 may have an arc-shaped rim, and the edges 201 and 202 may be formed into curved surfaces on the end of the pressure plate 20. The pressure points having a planar shape may be formed along the edges 201 and 202 having a curved surface shape so as to three-dimensionally extend in a region in which the edges come into contact with the back of the neck. The shape of the pressure points may be changed according to the thickness of the user's neck or the condition of the user's skin, for example.

The edges 201 and 202 include the first edge 201 which extends upward from the arch portion 110 and is configured to be interposed between the back of the neck and the arch portion (see FIG. 7) and the second edge 202 which extends outward of the first edge 201 and is disposed higher than the first edge 201. That is, as described above, one or more edges 201 and 202 may be formed on each pressure plate 20, and when two edges 201 and 202 are provided, the first edge 201 and the second edge 202 may be successively formed. In the embodiment of the present invention, as described above, each of the second cervical vertebra pressure plate 22, the third cervical vertebra pressure plate 23, the fourth cervical vertebra pressure plate 24, and the fifth cervical vertebra pressure plate 25 is formed with both the first edge 201 and the second edge 202. In addition, as described above, the first cervical vertebra pressure member 21 includes the pressure portion 21a formed into a curved surface. The first cervical vertebra pressure member 21, which is smaller than the pressure plates 20, includes the single pressure portion 21a in the form of a curved surface.

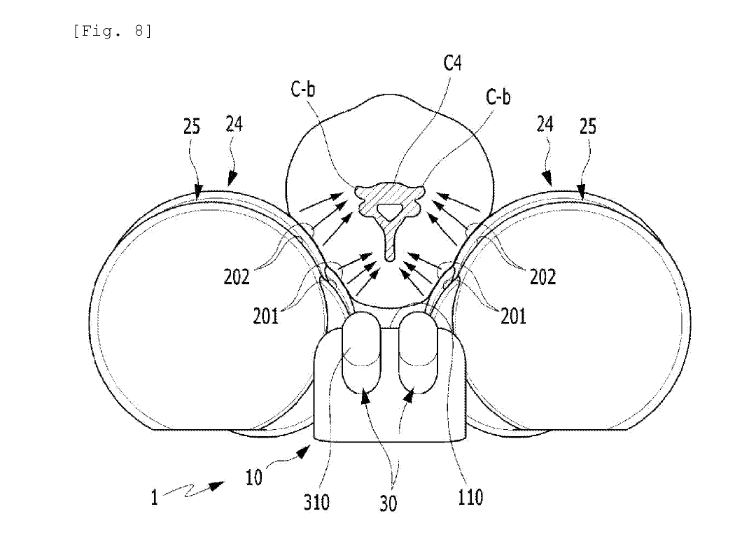

When the two edges 201 and 202 are provided, as described above, it is possible to three-dimensionally apply stimulus using the first edge 201 and the second edge 202 having different heights. That is, the first edge 201, which has a lower height, may be adjacent to the spinous process of the cervical vertebrae to transfer pressure to the spinous process, and the second edge 202, which has a higher height, may be adjacent to the transverse process of the cervical vertebrae to transfer pressure to the transverse process (see FIG. 8). By forming the two different edges 201 and 202 on the single pressure plate as described above, it is possible to very easily transfer pressure in the longitudinal and transverse directions of the cervical vertebrae, for example. Both the first edge 201 and the second edge 202, which are formed into curved surfaces, may have different centers of curvature. The first edge 201 may be formed into a curved surface which is shorter than the second edge 202. This will be described below in more detail.

For example, the height and the arrangement of the edges 201 and 202 may be changed according to, for example, the height and arrangement of the pressure plates 20 and the arrangement of the first cervical vertebra pressure member 21 which transfers pressure along with the pressure plates 20. Thus, the arrangement of the pressure plates 20 and the first cervical vertebra pressure member 21 will be described below in more detail. As described above, the pressure plates 20 include the second cervical vertebra pressure plate 22, the third cervical vertebra pressure plate 23, the fourth cervical vertebra pressure plate 24, and the fifth cervical vertebra pressure plate 25. In the pressure plates 20 and the first cervical vertebra pressure member 21, as illustrated in FIG. 4, the uppermost end of the fourth cervical vertebra pressure plate 24 may be located higher than the uppermost end of the fifth cervical vertebra pressure plate 25, the uppermost end of the second cervical vertebra pressure plate 22 may be located lower than the uppermost end of the third cervical vertebra pressure plate 23, and the uppermost end of the first cervical vertebra pressure plate 21 may be located lower than the uppermost end of the second cervical vertebra pressure plate 22.

Due to this arrangement, the pressure plates 20 and the first cervical vertebra pressure member 21 may form a structure, the height of which increases from the fifth cervical vertebra pressure plate 25 toward the fourth cervical vertebra pressure plate 24 and the third cervical vertebra pressure plate 23 and then decreases toward the second cervical vertebra pressure plate 22 and the first cervical vertebra pressure member 21. The uppermost ends of the fourth cervical vertebra pressure plate 24 and the third cervical vertebra pressure plate 23 may have the same height, but are not necessarily limited thereto. In some cases, the heights of the uppermost ends of the fourth cervical vertebra pressure plate 24 and the third cervical vertebra pressure plate 23 may be appropriately adjusted so as to be the same as or be different from each other.

The above-described structure may correspond to the shape of the arch portion 110. As described above, since the plurality of pressure plates 20 are connected to opposite sides of the arch portion 110 and are arranged along the arch portion 110, the pressure plates 20 may be arranged so that the height thereof is changed to correspond to the convexly protruding shape (i.e., an arched shape) of the arch portion 110. With this structure, the plurality of pressure plates 20 may effectively come into contact with the back of the neck along the curved cervical vertebrae. In addition, the edges 201 and 202 formed on the pressure plates 20 may form pressure points, the heights of which are also changed to correspond to the curved cervical vertebrae, so as to three-dimensionally transfer pressure. If necessary, the height of the pressure plates 20 may be adjusted independently of the arch portion 110 by changing the width of the pressure plates 20, for example.

In particular, in the case of the pressure plates 20 (the second to fifth cervical vertebra pressure plates) formed with both the first edge 201 and the second edge 202, the position of the second edge 202 having a higher height may be changed to correspond to the height of the pressure plates 20. That is, the height of the second edge 202, which is located at a higher height on each pressure plate 20, may be changed to correspond to the structure in which the uppermost end of the fourth cervical vertebra pressure plate 24 is located higher than the uppermost end of the fifth cervical vertebra pressure plate 25 and the uppermost end of the second cervical vertebra pressure plate 22 is located lower than the uppermost end of the third cervical vertebra pressure plate 23 in the direction in which the arch portion 110 protrudes. Hereinafter, for example, the arrangement of the edges 201 and 202 corresponding to the arrangement of the pressure plates 20 and the shape of the edges 201 and 202 will be described in more detail with reference to FIGS. 5 and 6.

FIG. 5 is a cross-sectional view of the cervical vertebra correction device of FIG. 1, and FIG. 6 is a view illustrating one of the pressure plates of the cervical spine brace of FIG. 1.

As illustrated in FIG. 5, in the cervical spine brace 1 according to the embodiment of the present invention, the uppermost end of the fourth cervical vertebra pressure plate 1 is located higher than the uppermost end of the fifth cervical vertebra pressure plate 25, and the uppermost end of the second cervical vertebra pressure plate 22 is located lower than the uppermost end of the third cervical vertebra pressure plate 25. The height of the second edge 202 is changed to correspond to this structure. That is, as illustrated, the second edge 202 of the fourth cervical vertebra pressure plate 24 is located higher than the second edge 202 of the fifth cervical vertebra pressure plate 25, and the second edge 202 of the second cervical vertebra pressure plate is located lower than the second edge 202 of the third cervical vertebra pressure plate 23. The heights of the second edge 202 of the fourth cervical vertebra pressure plate 24 and the second edge 202 of the third cervical vertebra pressure plate 23 may be the same as or different from each other according to the arrangement of the fourth cervical vertebra pressure plate 24 and the third cervical vertebra pressure plate 23 described above. In this way, it is possible to form pressure points, the heights of which are three-dimensionally changed to correspond to the curved cervical vertebrae. In addition, since the pressure portion 21a, having a curved surface shape, of the first cervical vertebra pressure member 21 is located lower than the second edges 202 of the fifth cervical vertebra pressure plate 25, the fourth cervical vertebra pressure plate 24, the third cervical vertebra pressure plate 23, and the second cervical vertebra pressure plate 22, it is possible to form pressure points, the heights of which are three-dimensionally changed to correspond to the curved cervical vertebrae based on the above-described structure. The first edges 201 formed on the respective pressure plates 20 and the pressure portion 21a of the first cervical vertebra pressure member 21 have different lengths to three-dimensionally transfer pressure. As described above, the first edge 201 is an edge that is formed into a curved surface extending upward from the arch portion 110, and similarly, the pressure portion 21a is formed into a curved surface extending upward from the arch portion 110. The length of the curved surface is greatest at the first cervical vertebra pressure member 21. That is, the curved surface forming the pressure portion 21a is longer than the edge (i.e., the first edge 201) which is formed into a curved surface extending upward from the arch portion 110. As illustrated in FIG. 5, the first edges 201 may be arranged such that the length thereof gradually increases from the fifth cervical vertebra pressure plate 25 toward the second cervical vertebra pressure plate 22, and the length of the curved surface may be the greatest at the pressure portion 21a formed on the first cervical vertebra pressure member 21. This arrangement may be more effective since the curved surfaces of the first edges 201 and the pressure portion 21a may be located lower than the above-described second edges 202 and may come into wider contact with the back of the neck than the second edges. In particular, since the length of the curved surface is the greatest at the first cervical vertebra pressure member 21 that is closest to the head, the first cervical vertebra pressure member may more stably come into contact with the back of the neck to transfer pressure using the curved surface.

In addition, at least one of the gaps between the respective pressure plates 20 and the gap between the pressure plate 20 and the first cervical vertebra pressure member 21 may be different from another gap. That is, at least one of the distance between the first cervical vertebra pressure member 21 and the second cervical vertebra pressure plate 22, the distance between the second cervical vertebra pressure plate 22 and the third cervical vertebra pressure plate 23, the distance between the third cervical vertebra pressure plate 23 and the fourth cervical vertebra pressure plate 24, and the distance between the fourth cervical vertebra pressure plate 24 and the fifth cervical vertebra pressure plate 25 may be greater or smaller than another distance. The arrangement based on these distances may be adjusted to correspond to the arrangement of the respective cervical vertebrae. For example, the distances between the respective pressure plates 20 and the distance between the pressure plate 20 and the first cervical vertebra pressure member 21 may be appropriately adjusted to more effectively transfer pressure in consideration of the distances between the first to seventh cervical vertebrae and the lengths of the spinous process and the transverse process formed on the cervical vertebrae, for example.

The pressure plates 20 are connected in the form of a cantilever to the support block 10, as illustrated in FIGS. 3, 5 and 6. Thus, a portion of the pressure plate which is not connected to the support block 10 may form a free end so as to be appropriately deformed. In addition, the first cervical vertebra pressure member 21 may also formed into a plate shape and may protrude in the form of a cantilever from the support block 10. However, as in another embodiment which will be described below, the first cervical vertebra pressure member 21 is not limited to a plate shape, and the shape or location thereof may be changed differently from the pressure plates 20. The pressure plates 20 or the first cervical vertebra pressure member 21 may be elastically deformed and the limit of the elastic deformation may also be adjusted by appropriately selecting the material of the pressure plates 20, the material of the first cervical vertebra pressure member 21, the material of the support block 10, or the material of connection portions between the pressure plates 20 and the support block 10 and between the first cervical vertebra pressure member 21 and the support block 10. Thus, for example, even when the user inserts the pressure target region (i.e., the back of the neck) between the pressure plates 20 by applying excessive pressure, the pressure plates 20 or the first cervical vertebra pressure member may be appropriately deformed according to the insertion of the pressure target region, thereby absorbing shock. In addition, the pressure plates or the first cervical vertebra pressure member may be appropriately deformed according to the weight of the user's head, for example, so that the distance between the respective pressure plates 20 or the distance between the pressure plate 20 and the first cervical vertebra pressure member 21 may also be changed. Accordingly, it is possible to more naturally transfer pressure to the back of the neck of the user.

As illustrated in FIG. 6, the first edge 201 and the second edge 202 are formed into curved surfaces having different centers of curvature a and b. The following exemplary description related to the fifth cervical vertebra pressure plate 25 with reference to FIG. 6 is also equally applicable to the second to fourth cervical vertebra pressure plates each having the first edge 201 and the second edge 202. The first edge 201 and the second edge 202 are formed into curved surfaces to more easily come into contact with the three-dimensional curved surface of the neck and the periphery thereof and transfer pressure thereto. In particular, the first edge 201 and the second edge 202 are formed into curved surfaces, the centers of curvature a and b of which are spaced apart from each other. As such, even if the first edge and the second edge are formed into curved surfaces having the same curvature, the first edge and the second edge may apply pressure to different positions on the back of the neck.

The first edge 201 and the second edge 202 may be formed into curved surfaces connected to each other, and the shape of the curved surfaces may be appropriately adjusted by changing the centers of curvature a and b and the radii of curvature thereof as needed. Thus, the first edge and the second edge may form a structure capable of easily transferring pressure in various directions to, for example, the spinous process and the transverse process of the cervical vertebrae via the different curved surfaces thereof. A curved surface capable of concentrating pressure on a smaller area to transfer the pressure thereto is formed when the radius of curvature thereof increases, whereas a curved surface capable of uniformly transferring pressure to a wider area is formed when the radius of curvature thereof decreases. Therefore, when curved surfaces are arranged to have different centers of curvature, it is possible to form the edges 201 and 202 capable of three-dimensionally transferring pressure. Hereinafter, a pressure protrusion 30 will be described in detail with reference to FIGS. 1 to 3. The cervical spine brace 1 according to the embodiment of the present invention includes the pressure protrusion 30 which protrudes from the top of the arch portion 110, as illustrated in FIGS. 1 to 3. The pressure protrusion 30 may protrude from the top of the arch portion 110 to form a pressure point, which is wider than the edges 201 and 202, on the back of the neck that does not come into contact with the edges 201 and 202. A pair of pressure protrusions 30 may be formed as illustrated, and each may form a wide pressure point using a relatively wide contact surface 310. In particular, at least a portion of the respective pressure protrusions 30 may be disposed to correspond to the sixth cervical vertebra and the seventh cervical vertebra, and the pressure protrusions 30 may respectively include the inclined contact surface 310 to transfer pressure to the sixth cervical vertebra and the seventh cervical vertebra together.

That is, it is possible to form a structure capable of transferring pressure to the first to fifth cervical vertebrae using the pressure plates 20 and the first cervical vertebra pressure member 21 described above and also transferring pressure to the sixth and seventh cervical vertebrae using the pressure protrusions 30. In particular, the pressure protrusions 30 straightly protrude from the top of the arch portion 110 toward the back of the neck, thereby being capable of directly applying pressure to the sixth and seventh cervical vertebrae which are connected to the thoracic vertebrae. In this way, by forming the arch portion 110 protruding from the support block 10 toward the back of the neck, connecting the plurality of pressure plates 20 to the support block 10 along opposite sides of the arch portion 110, forming a pressure structure (the first cervical vertebra pressure member 21) protruding from the arch portion 110, and forming the pressure protrusions 30 on the top of the arch portion 110 of the support block 10 which are not connected to the pressure plates 20, it is possible to very easily apply pressure to the entire back of the neck and to apply more optimized stimulus to the cervical vertebrae and the periphery thereof from positions corresponding to the respective cervical vertebrae. The following description will be made with reference to FIGS. 7 to 8.

FIGS. 7 and 8 are views illustrating the use state of the cervical spine brace of FIG. 1.

FIG. 7 illustrates the neck of the user and the periphery thereof, along with the cervical spine brace, which are viewed from the lateral side, and in FIG. 7, the pressure plates of the cervical spine brace are illustrated in cross section in order to show the arrangement of the pressure plates. FIG. 8 illustrates the neck inserted between the pressure plates and exemplary positions of the cervical vertebrae inside the neck.

The cervical spine brace 1 according to the embodiment of the present invention is used as illustrated in FIGS. 7 and 8. As described above, the back of the neck of the user is inserted into the three-dimensional accommodating space defined between the pressure plates 20 and the arch portion 110 of the support block 10. The back of the neck of the user is maintained in a curved form to correspond to the arched shape of the support block 10 and stably maintains the posture thereof. In such a state, the back of the neck is naturally pressed and relaxed by the weight of the head, the weight of the upper body, and the tension and the weight of the neck connected to both the head and the upper body. In particular, as illustrated, the pressure plates 20 and the first cervical vertebra pressure member 21 are inclined in the direction in which the spinous process C-a of the corresponding cervical vertebra, i.e., the first cervical vertebra C1, the second cervical vertebra C2, the third cervical vertebra C3, the fourth cervical vertebra C4, and the fifth cervical vertebra CT extends to support the back of the neck. Accordingly, it is possible to more smoothly transfer pressure to the respective cervical vertebrae.

In addition, as illustrated in FIG. 7, the pressure protrusions 30 transfer pressure to both the sixth cervical vertebra C6 and the seventh cervical vertebra C7 using the contacts surfaces 310 thereof which are inclined downward from the head toward the back. Thus, appropriate pressure is transferred to the respective cervical vertebrae independently of each other. Since pressure is transferred to the respective cervical vertebrae, a facet joint which is provided between the respective cervical vertebrae to interconnect the cervical vertebrae may be naturally relaxed. That is, through the structure of applying pressure to the respective cervical vertebrae independently of each other, it is possible to concentrate correction force to the facet joint connected to the cervical vertebrae and to effectively correct the deformed or distorted shape of the cervical vertebrae.

In particular, as illustrated, since the user installs the cervical vertebra correction device 1 so as to be supported on the floor and uses the device 1 in a lying position, the back of the neck comes into contact with the edges 201 and 202 of the pressure plates 20 by the weight thereof so that pressure points are formed by the edges and the above-described relaxation point is formed between the pressure points (i.e., between the pressure plates 20). In addition, since the first cervical vertebra pressure member 21 also comes into contact with the back of the neck to form a pressure point, the above-described relaxation point may also be formed between the pressure plate 20 and the first cervical vertebrae pressure member 21. Since the body located at the relaxation points is supported in the direction of gravity and naturally generates pressure in an opposite direction, a three-point support structure including the body, the relaxation points, and pressure points is formed. Through the use of this structure, it is possible to naturally transfer pressure to the cervical vertebrae and the periphery thereof and to induce stimulus and relaxation of the cervical vertebrae and the connection portion thereof.

In addition, as illustrated in FIG. 8, the edges 201 and 202 of the pressure plates 20 may three-dimensionally transfer pressure to both the transverse process C-b and the spinous process C-a of the cervical vertebrae. Thus, even when the cervical vertebrae is three-dimensionally deformed, it is possible to transfer pressure in various directions including, for example, the transverse direction (the horizontal direction in FIG. 8) and the longitudinal direction (the vertical direction in FIG. 8) to effectively correct the cervical vertebrae. In particular, it is possible to correct deformation of the cervical vertebra in the transverse direction by transferring pressure to the transverse process C-b of the cervical vertebra using the second edges 202 and to correct deformation of the cervical vertebra in the longitudinal direction by transferring pressure to the spinous process C-a of the cervical vertebra using the first edges 201. Based on the fact that the respective pressure transfer directions may be three-dimensionally defined to cross each other, correction of the cervical vertebra may be realized by applying stimulus in various directions including the transverse and longitudinal directions described above.

In particular, it is possible to easily generate a longitudinal pressure component in FIG. 8 by applying pressure to opposite sides of the back of the neck using the first edge 201 which is formed into a curved surface at a relatively low position and the pressure portion (see “21a” in FIG. 5), and it is possible to easily generate a transverse pressure component in FIG. 8 by applying pressure to opposite sides of the back of the neck using the second edge 202 which is formed into a curved surface, having a center of curvature different from that of the first edge, at a relatively high position. That is, by transferring pressure in the direction perpendicular to the tangent of the curved surface and by using both the longitudinal pressure component and the transverse pressure component, it is possible to very easily correct, for example, three-dimensionally deformed cervical vertebrae. In addition, when pressure is transferred in various directions as described above, since muscles of the cervical vertebrae as well as the periphery thereof are stimulated together, it is possible to relax the tense muscles, thus effectively eliminating pain.

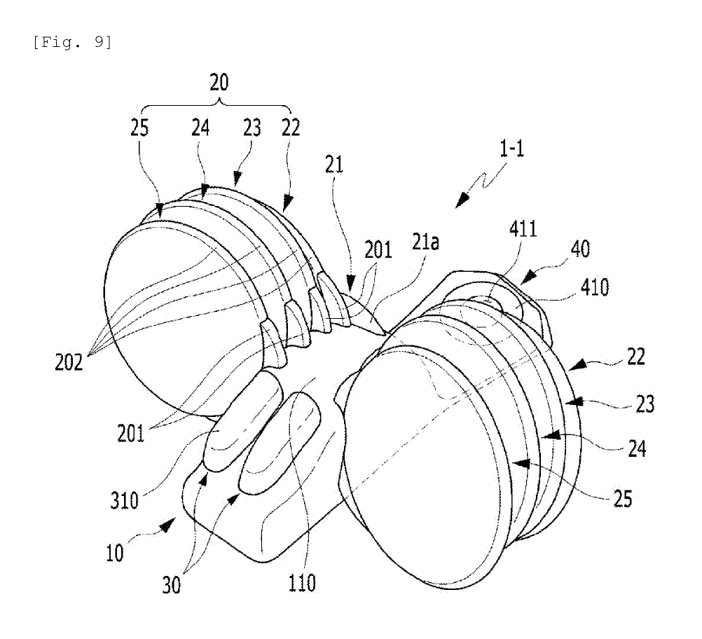

Hereinafter, a cervical spine brace according to another embodiment of the present invention will be described in detail with reference to FIGS. 9 to 11. Hereinafter, in order to provide a brief and clear description, the following description will concentrate only on parts different from those of the above-described embodiment, and a description related to unmentioned parts will be replaced with the above description.

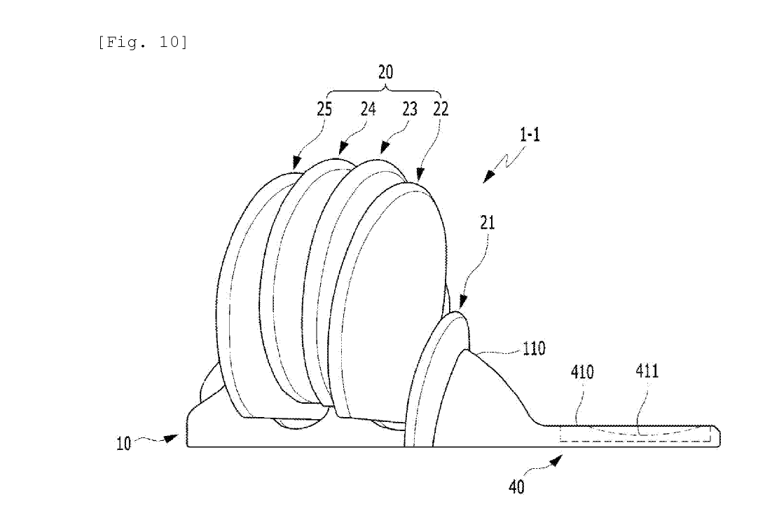

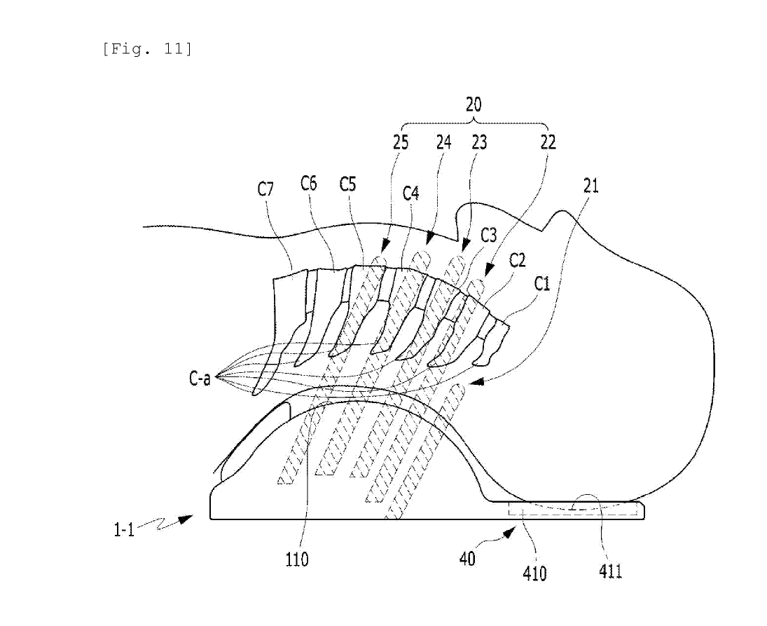

FIG. 9 is a perspective view of a cervical spine brace according to another embodiment of the present invention, FIG. 10 is a side view of the cervical spine brace of FIG. 9, and FIG. 11 is a view illustrating the use state of the cervical spine brace of FIG. 9.

Referring to FIGS. 9 and 10, the cervical spine brace 1-1 according to another embodiment of the present invention includes a headrest 40 connected to the support block 10. As illustrated in FIGS. 9 and 10, the headrest 40 may be formed so as to extend in a given direction from the support block 10. The headrest 40 may be integrally formed with the support block 10, or may be separately formed and connected to the support block. In addition, the support block 10 and the headrest 40 may be detachably coupled to each other using a groove structure, a protruding structure, or a slidable insertion structure, for example, as needed. The headrest 40 is connected and stably fixed to the support block 10.

The headrest 40 has a seating recess 411 in which a rear portion of the user's head (i.e., the back of the head) is accommodated. The seating recess 411 may be formed in a recessed shape to accommodate a portion of the back of the head therein. Thus, as illustrated in FIG. 11, more stable correction may be implemented in the state in which the user's head is seated on the headrest 40. As described above, through the use of the pressure plates 20 three-dimensionally connected to the support block 10, the edges 201 and 202 formed on the pressure plates 20, the first cervical vertebra pressure member 21 having the pressure portion (see “21a” in FIG. 9), and the pressure protrusions 30 formed on the arch portion 110, for example, it is possible to three-dimensionally transfer pressure to the cervical vertebrae and the periphery thereof of the user to correct the cervical vertebrae and the periphery thereof. At this time, by adjusting the position of the user's head using the headrest 40, it is possible to cause the back of the neck to more effectively come into contact with the pressure plates 20.

In particular, the cervical spine brace 1-1 according to another embodiment of the present invention may include a support pad 410 detachably coupled to a head contact portion of the headrest 40. As illustrated in FIGS. 9 and 10, the support pad 410 may be formed in the head contact portion, such as the inside of the headrest 40, so as to be detachably coupled thereto. Any one of different shapes of support pads 410 may be selectively coupled, or one or more support pads may be coupled to overlap each other. When various shapes of support pads 410 are provided as described above and a portion of the cervical spine brace, to which the support pad 410 is coupled, i.e., the head contact portion is changed in shape, increased user correction effects may be achieved. When the shape of the head contact portion is changed, for example, the shape, size and height of the seating recess 411 formed in the contact portion may be appropriately changed. Thus, more effective correction may be exerted by adjusting the position of the user's head. With this structure, it is possible to transfer optimum stimulus to each region of the cervical vertebrae, thereby achieving more effective correction and very easily eliminating muscle pain, for example.

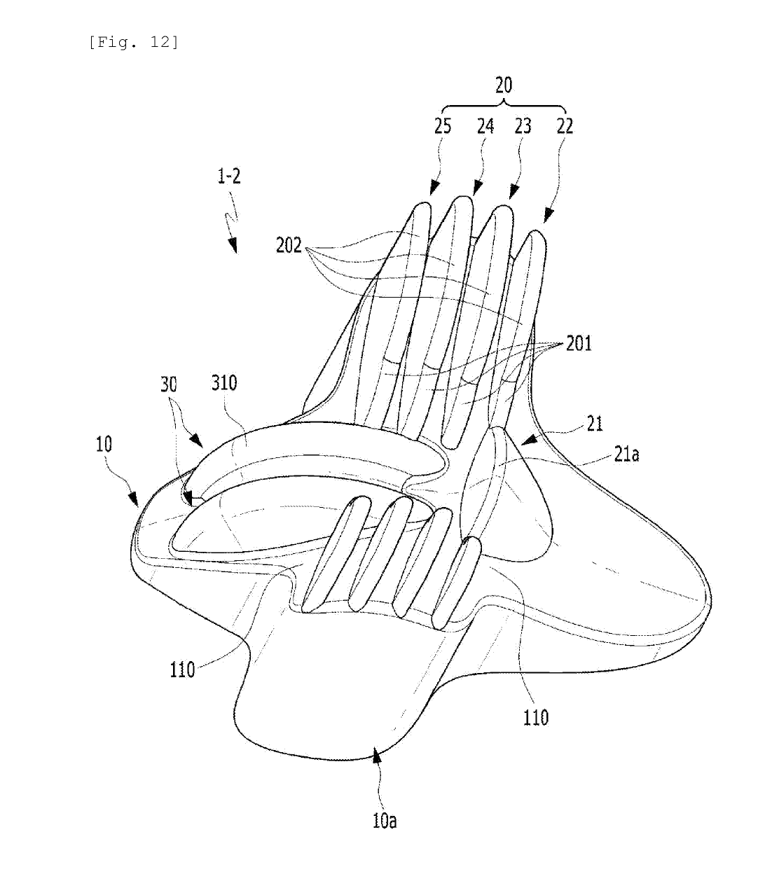

Hereinafter, a cervical spine brace according to a further embodiment of the present invention will be described in detail with reference to FIGS. 12 to 14. Hereinafter, in order to provide a brief and clear description, the following description will concentrate only on parts different from those of the above-described embodiment and a description related to unmentioned parts will be replaced with the above description.

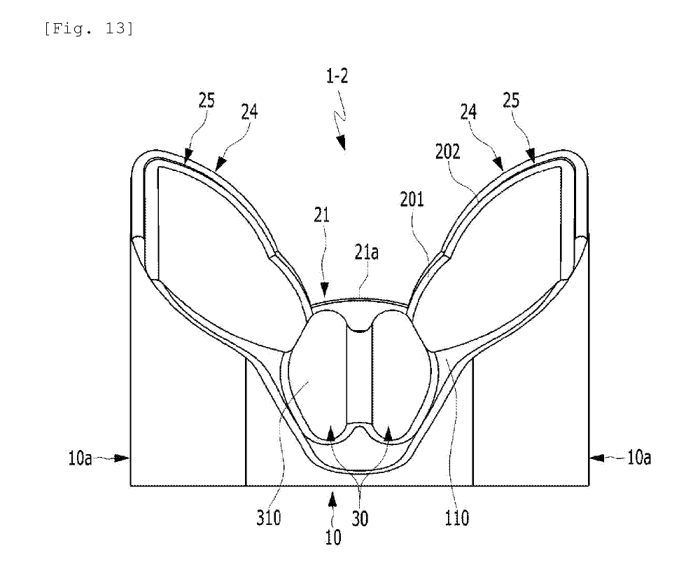

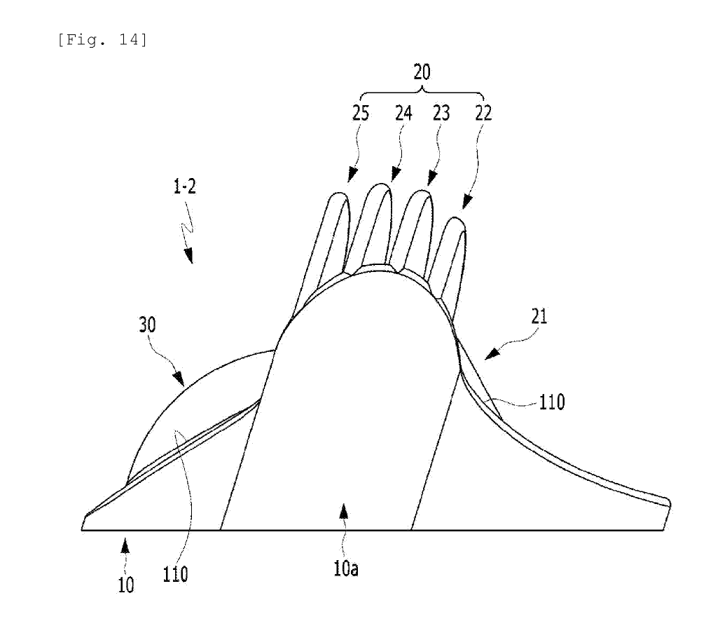

FIG. 12 is a perspective view of a cervical spine brace according to a further embodiment of the present invention, FIG. 13 is a front view of the cervical spine brace of FIG. 12, and FIG. 14 is a side view of the cervical spine brace of FIG. 12.

Referring to FIGS. 12 to 14, the cervical spine brace 1-2 according to a further embodiment of the present invention includes a connection block 10a which interconnects and secures the lower ends of the pressure plates 20, and at least a portion of the first cervical vertebra pressure member 21 extends between a pair of the second cervical vertebra pressure plates 22. In addition, at least a portion of the pressure protrusion 30 extends between pairs of the pressure plates 20. In a modification, an outer portion of the pressure plate 20 may be formed into a cut shape so as to be integrated with the connection block 10a (see FIG. 13), but the arrangement of the first edge 201 and the second edge 202 is the same as the above description. That is, the cervical spine brace 1-2 is configured to more firmly support the pressure plates 20 by the connection block 10a while maintaining the pressure effect of the edges as described above.

The connection block 10a interconnects the lower ends of the pressure plates 20. The lower end of each pressure plate 20 may include a portion that is connected to the support block 10, and the connection block 10a may be formed as a structure that is connected to the lower end of the pressure plate 20 to support the pressure plate 20. The connection block 10a may be formed into an expanded form from the support block 10 and may be integrally formed with the support block 10. Thus, the structure in which the support block 10 and the pressure plates 20 are firmly connected to each other and the pressure plates 20 are connected to each other and are firmly secured is realized by the connection block 10a. In addition, the area over which the cervical spine brace 1-2 is in contact with the floor may be increased by the connection block 10a which is expanded outward from the support block 10. The shape of the connection block 10a is not necessarily limited to the illustrated shape, and may be modified into various shapes as long as the connection block interconnects and secures the lower ends of the pressure plates 20. Through the provision of the connection block 10a, it is possible to more effectively interconnect the pressure plates 20 and to increase the structural stability of the cervical spine brace 1-2.

In addition, as illustrated in FIGS. 12 to 14, the first cervical vertebra pressure member 21 and the pressure protrusions 30 may be appropriately changed in shape. In particular, the first cervical vertebra pressure member 21 may have a wide protrusion shape, rather than a plate shape, and may protrude upward from the arch portion 110. The first cervical vertebra pressure member 21 may be provided in singular, rather than being provided in a pair. As illustrated, at least a portion of the first cervical vertebra pressure member 21 may extend between a pair of the second cervical vertebra pressure plates 22 and may come into contact with the back of the neck. In addition, at least a portion of each pressure protrusion 30 may extend between the pairs of pressure plates 20 to greatly increase the area of the contact surface 310. Thereby, it is possible to increase the area over which the pressure protrusions 30 and the first cervical vertebra pressure member 21 come into contact with the back of the neck of the user and to more effectively apply pressure to a region of the back of the neck that does not come into contact with the pressure plates 20.

The cervical vertebra correction device 1-2 having the above-described structure may transfer stimulus to respective regions of the cervical vertebrae and effectively correct the cervical vertebrae. In addition, the structural stability of the cervical vertebra correction device 1-2 may be greatly increased.

Although the embodiments of the present invention have been described above with reference to the accompanying drawings, it will be understood by those skilled in the art to which the embodiments pertain that the present invention may be implemented in other forms without changing the technical sprit or essential features thereof. Therefore, the embodiments described above should be understood to be exemplary in all terms, but should not be understood to be limitative.

### DESCRIPTION OF REFERENCE NUMERALS

1, 1-1, 1-2: cervical spine brace
10: support block
10a: connection block
20: pressure plate
21: first cervical vertebra pressure member
21a: pressure portion
22: second cervical vertebra pressure plate
23: third cervical vertebra pressure plate
24: fourth cervical vertebra pressure plate
25: fifth cervical vertebra pressure plate
30: pressure protrusion
40: headrest
110: arch portion
201: first edge
202: second edge
310: contact surface
410: support pad
411: seating recess
a, b: center of curvature
C1: first cervical vertebra
C2: second cervical vertebra
C3: third cervical vertebra
C4: fourth cervical vertebra
C5: fifth cervical vertebra
C6: sixth cervical vertebra
C7: seventh cervical vertebra
C-a: spinous process
C-b: transverse process

### INDUSTRIAL APPLICABILITY

The present invention is capable of transferring optimum stimulus to each region of the cervical vertebrae to realize more effective correction, and thus has industrial applicability.

1. A cervical spine brace comprising:
a support block configured to be disposed on a back of a neck between a head and a back and having an arch portion corresponding to an arch of the back of the neck; and
a plurality of pressure plates connected in a pair to opposite sides of the arch portion of the support block and configured to be inclined from the support block toward the back of the neck, each pressure plate having at least one edge disposed on an end thereof to form pressure points at different positions on the back of the neck according to a height of the arch portion.

2. The cervical spine brace according to claim 1, wherein the arch portion includes a curved surface that is convex toward the back of the neck.

3. The cervical spine brace according to claim 1, wherein at least a portion of each pressure plate is formed higher than the arch portion so that the back of the neck is inserted between the pressure plate and the arch portion.

4. The cervical spine brace according to claim 3, wherein the edge is located on the end of the pressure plate located higher than the arch portion.

5. The cervical spine brace according to claim 1, wherein at least a portion of each pressure plate has an arch-shaped rim, and the edge is formed into a curved surface on the end of the pressure plate.

6. The cervical spine brace according to claim 1, wherein the at least one edge includes a first edge that extends upward from the arch portion and is interposed between the back of the neck and the arch portion and a second edge that extends outward of the first edge and is located higher than the first edge.

7. The cervical spine brace according to claim 6, wherein the first edge is disposed adjacent to a spinous process of a cervical vertebra to be able to transfer pressure to the spinous process, and the second edge is disposed adjacent to a transverse process of the cervical vertebra to be able to transfer pressure to the transverse process.

8. The cervical spine brace according to claim 6, wherein the first edge and the second edge are formed respectively into curved surfaces having different centers of curvature.

9. The cervical spine brace according to claim 6, wherein the first edge is formed into a curved surface that is shorter than the second edge.

10. The cervical spine brace according to claim 1, further comprising a first cervical vertebra pressure member having at least a portion disposed at a position corresponding to a first cervical vertebra, the first cervical vertebra pressure member protruding from the support block,
wherein the pressure plates include a second cervical vertebra pressure plate disposed at a position corresponding to a second cervical vertebra, a third cervical vertebra pressure plate disposed at a position corresponding to a third cervical vertebra, a fourth cervical vertebra pressure plate disposed at a position corresponding to a fourth cervical vertebra, and a fifth cervical vertebra pressure plate disposed at a position corresponding to a fifth cervical vertebra, and
wherein the first cervical vertebra pressure member has a size smaller than a size of the pressure plates.

11. The cervical spine brace according to claim 10, wherein the fourth cervical vertebra pressure plate has an uppermost end located higher than an uppermost end of the fifth cervical vertebra pressure plate, the second cervical vertebra pressure plate has an uppermost end located lower than an uppermost end of the third cervical vertebra pressure plate, and the first cervical vertebra pressure member has an uppermost end located lower than the uppermost end of the second cervical vertebra pressure plate.

12. The cervical spine brace according to claim 10, wherein the first cervical vertebra pressure member has a pressure portion formed into a curved surface that extends upward from the arch portion, and each of the second cervical vertebra pressure plate, the third cervical vertebra pressure plate, the fourth cervical vertebra pressure plate, and the fifth cervical vertebra pressure plate has two or more edges including one edge formed into a curved surface that extends upward from the arch portion and another edge formed into another curved surface.

13. The cervical spine brace according to claim 12, wherein the curved surface forming the pressure portion has a length greater than a length of the one edge formed into the curved surface that extends upward from the arch portion.

14. The cervical spine brace according to claim 10, wherein at least one of a distance between the first cervical vertebra pressure member and the second cervical vertebra pressure plate, a distance between the second cervical vertebra pressure plate and the third cervical vertebra pressure plate, a distance between the third cervical vertebra pressure plate and the fourth cervical vertebra pressure plate, and a distance between the fourth cervical vertebra pressure plate and the fifth cervical vertebra pressure plate is greater or smaller than another distance.

15. The cervical spine brace according to claim 10, wherein at least a portion of the first cervical vertebra pressure member extends between a pair of the second cervical vertebra pressure plates.

16. The cervical spine brace according to claim 1, wherein each pressure plate is inclined in a direction corresponding to a direction in which a spinous process of a cervical vertebra extends.

17. The cervical spine brace according to claim 1, wherein each pressure plate is connected in a cantilever form to the support block.

18. The cervical spine brace according to claim 1, further comprising a pressure protrusion that protrudes from a top of the arch portion and is able to form a pressure point wider than the edge on the back of the neck that does not come into contact with the edge.

19. The cervical spine brace according to claim 18, wherein the pressure protrusion has at least a portion disposed at a position corresponding to a sixth cervical vertebra and a seventh cervical vertebra, and includes an inclined contact surface to transfer pressure to both the sixth cervical vertebra and the seventh cervical vertebra via the contact surface.

20. The cervical spine brace according to claim 18, wherein the pressure protrusion has at least a portion that extends between a pair of the pressure plates.

21. The cervical spine brace according to claim 1, further comprising a headrest connected to the support block.

22. The cervical spine brace according to claim 21, further comprising a support pad detachably coupled to a head contact portion of the headrest.

23. The cervical spine brace according to claim 1, further comprising a connection block configured to interconnect and secure lower ends of the pressure plates.
